(19) United States
(12) Patent Application Publication
(10) Pub. No.: US 2024/0335113 A1
(43) Pub. Date: Oct. 10, 2024

(54) WIRELESS HEALTH MONITORING DEVICES AND METHODS OF MANAGING ENERGY CONSUMPTION OF SUCH DEVICES

(71) Applicant: KONINKLIJKE PHILIPS N.V., Eindhoven (NL)

(72) Inventors: Marco Mamprin, Eindhoven (NL); Hendrik Huijgen, Eindhoven (NL); Ewout Brandsma, Eindhoven (NL); Warner Rudolph Theophile Ten Kate, Eindhoven (NL); Wilhelmus Johannes Joseph Stut, EIndhoven (NL)

(21) Appl. No.: 18/617,815

(22) Filed: Mar. 27, 2024

Related U.S. Application Data
(60) Provisional application No. 63/456,902, filed on Apr. 4, 2023.

(51) Int. Cl. A61B 5/00

(52) U.S. Cl. CPC A61B 5/0006; A61B 5/0022; A61B 2560/0219

(57) ABSTRACT

The present disclosure is directed to wireless health monitoring devices adapted for intermittent data transmission with controlled energy consumption as well as methods of managing energy consumption by a wireless health monitoring device. As described herein, the devices and methods eliminate the need for an intermediating electronic device, enable direct uploading of health-related data to a back-end device, improve useability of the wireless health monitoring device, reduce costs associated with the wireless health monitoring device, increase the efficiency of data transmission from the health monitoring device, and improve the lifespan of the device's energy source. The devices and methods of the present disclosure find particular application in clinical, out-of-hospital patient monitoring, although other applications are contemplated.

### CROSS-REFERENCE TO RELATED APPLICATIONS

This patent application claims the priority benefit under 35 U.S.C. § 119 (c) of U.S. Provisional Application No. 63/456,902, filed on Apr. 4, 2023, the contents of which are herein incorporated by reference.

### FIELD OF THE DISCLOSURE

The present disclosure relates generally to wireless health monitoring devices and methods of operating such wireless health monitoring devices, and more specifically to wireless health monitoring devices adapted for intermittent data transmission and to methods of managing the energy consumption by such devices.

### BACKGROUND

Continuous advancements in physiological sensor electronics, energy storage devices, telecommunications, and related technologies have enabled a growing landscape of wireless health monitoring devices. As a result, wireless health monitoring devices, including wearable devices such as wrist bracelets, watches, rings, patches, and the like, have become an increasingly viable option for out-of-hospital monitoring of patients. For example, Koninklijke Philips N. V. offers a mobile cardiac outpatient telemetry (MCOT) device, which uses a wearable patch that can be worn for up to 14 days and enables automatic detection and reporting of abnormal cardiovascular conditions.

However, many technical challenges still exist, especially for wireless devices intended for monitoring of ambulatory patients (i.e., patients who undergo monitoring outside the hospital while moving around and going about their day). In particular, these monitoring devices must be comfortably worn by patients and/or should not impede their normal daily activities. Thus, such devices are usually constrained in size, shape, and interface means. Further, such health monitoring devices are intended for clinical applications and, therefore, have heightened data privacy, data handling, and data quality requirements. For example, in order to obtain clinically-relevant health-related data, wireless health monitoring devices must include suitable physiological sensing capabilities as well as be able to reliably share the health-related data at clinically-appropriate times. Put another way, these wireless health monitoring devices must be able to share the health-related data they collected, reliably and in time to support clinical decision-making, regardless of the location of the monitoring device.

In some existing solutions, a health monitoring device (such as the MCOT device offered by Koninklijke Philips N. V.) is configured to operate while paired with a local intermediating device, such as a smartphone, tablet, or other mobile electronic device having its own energy source and more powerful telecommunication capabilities. In these systems, the health monitoring device may collect health-related data and utilize a low-power communications method to transfer the data to the local intermediating device, which then uses its own energy source to complete the transfer of the data to a target destination, such as a back-end server, where clinical decision-makers can access, analyze, and/or address issues detected based on the health-related data. This preserves the capacity and extends the lifetime of the energy source of the wireless health monitoring device and enables frequent uploads of the collected health-related data from the device. However, relying on a local intermediating device adds significant cost to the overall system and introduces additional complexity to the data sharing protocols and energy consumption management of the system. Further, the need for an additional device reduces the useability and convenience of associated the wireless health monitoring device by adding a second device that the patient needs to keep in proximity of the health monitoring device and separately maintain.

### SUMMARY OF THE DISCLOSURE

The present disclosure is directed to wireless health monitoring devices adapted for intermittent data transmission with controlled energy consumption and to methods of managing energy consumption by a wireless health monitoring device. As described herein, the “direct-to-cloud” devices and methods eliminate the need for an intermediating electronic device, enable direct uploading of health-related data (such as ECG data) to a back-end device, improve useability of and reduce costs associated with the wireless health monitoring device, increase the efficiency of data transmission from the health monitoring device, and improve the lifespan of the device's energy source. The devices and methods of the present disclosure find particularly suitable applications in clinical, out-of-hospital and ambulatory patient monitoring, although other applications are contemplated.

According to some embodiments and implementations of the present disclosure, a wireless health monitoring device adapted for intermittent data transmission with controlled energy consumption is provided. The device may include: a data module configured to generate and/or collect health-related data associated with at least one user over a period of time; a communication unit operatively connected to the data module, the communication unit being configured to wirelessly transmit health-related data over a network to a remote destination; a constrained energy source operatively connected to the data module and the communication unit for storing and providing electrical energy thereto; a controller operatively connected to the data module, the constrained energy source, and the communication unit, wherein the controller comprises a memory storing machine readable instructions and one or more processors that, when executing the machine-readable instructions, are configured to perform a pre-transmission assessment operation comprising one or more of the following: (i) determine whether a data package comprising the health-related data is ready to be transmitted from the device; (ii) determine an amount of energy available for the transmission of the data package within the constrained energy source; (iii) extract one or more network parameters having predictive value related to energy consumption by the device based on current network conditions; and (iv) determine whether to proceed with the transmission of the data package based on the amount of energy available for the transmission and the one or more network parameters.

In an aspect, the data module includes a physiological sensor associated with the user and the health-related data includes physiological sensor data collected by the physiological sensor. The physiological sensor may include an electrocardiogramansor configured to be worn by the user.

In an aspect, the constrained energy source may include a battery.

In an aspect, the one or more processors may be further configured to wirelessly transmit the data package from the device via the communication unit over the network or to delay the transmission based on an outcome of the pre-transmission assessment operation.

In an aspect, the pre-transmission assessment operation may include predicting an amount of energy necessary for the transmission of the data package based on the one or more network parameters; and wherein an outcome of the pre-transmission assessment operation includes generating instructions to the one of more processors to proceed with the transmission of the data package when the predicted amount of energy is less than or equal to an energy threshold, or to delay the transmission of the data package when the predicted amount of energy exceeds the energy threshold, and wherein the energy threshold is based at least in part on the amount of energy available for the transmission of the data package within the constrained energy source.

In an aspect, the energy threshold may be further based at least in part on an acuity level of the health-related data in the data package.

In an aspect, the amount of energy necessary for the transmission of the data package may be predicted based on the one or more network parameters using a trained machine learning model, the trained machine learning model comprising one or more of a support vector machine, a decision tree, a gradient boosted decision tree, a bagged tree, a logistic regression, and a neural network.

In an aspect, the one or more processors may be configured to extract the one or more network parameters having predictive value related to energy consumption by the device based on current network conditions by: (i) connecting the health monitoring device to the wireless network via the communication unit; and (ii) determining the one or more parameters based on the network conditions detected after connecting the health monitoring device to the wireless network; wherein the one or more parameters include one or more of a signal strength, a signal quality, a duration of the connecting step, and an amount of energy consumed by the device during the connecting step.

In an aspect, the transmission readiness determination for the data package comprising the health-related data may be based on one or more predetermined conditions, and the one or more processors may be further configured to activate the communication unit and initiate the pre-transmission assessment operation when the one or more predetermined conditions are met.

According to other embodiments and implementations of the present disclosure, a method of managing energy consumption by a wireless health monitoring device adapted for intermittent data transmission and comprising a data module, a controller having a memory and one or more processors, a communication unit, and a constrained energy source is provided. The method may include: determining, via the one or more processors of the controller, an amount of energy available from the energy source for transmission of a data package comprising health-related data; extracting, via the communication unit and the one or more processors of the controller, one or more network parameters having predictive value related to energy consumption by the device based on current network conditions; analyzing, via the one or more processors of the controller, the one or more network parameters to predict an amount of energy needed for the transmission of the data package using a trained machine learning model; determine whether to proceed with the transmission of the data package; and generating instructions to the one or more processors to proceed with the transmission of the data package when the predicted amount of energy is less than or equal to an energy threshold, or to postpone the transmission of the data package when the predicted amount of energy exceeds the energy threshold, wherein the energy threshold is based at least in part on the amount of energy available for the transmission of the data package within the constrained energy source.

In an aspect, the method can further include wirelessly transmitting the data package to a remote destination, or delaying the transmission of the data package by at least a first wait time.

In an aspect, the trained machine learning model may include one or more of a support vector machine, a decision tree, a gradient boosted decision tree, a bagged tree, a logistic regression, and a neural network.

In an aspect, the step of extracting the one or more network parameters having predictive value related to energy consumption by the device based on current network conditions may include: connecting the health monitoring device to a wireless network via the communication unit; and determining the one or more parameters based on the network conditions detected after connecting the health monitoring device to the wireless network; wherein the one or more parameters include one or more of a signal strength, a signal quality, a duration of the connecting step, and an amount of energy consumed by the device during the connecting step.

In an aspect, the step of determining the one or more parameters may include transmitting a portion of the data package via the wireless network and detecting the network conditions during said transmission.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is an illustration of a health monitoring system according to certain aspects of the present disclosure.

FIG. 2A is a block diagram illustrating a health monitoring device adapted for intermittent data transmission according to aspects of the present disclosure.

FIG. 2B is a block diagram illustrating a controller of a health monitoring device according to aspects of the present disclosure.

FIG. 3A is a flowchart illustrating a method of managing energy consumption by a wireless health monitoring device adapted for intermittent data transmission according to aspects of the present disclosure.

FIG. 3B is a flowchart illustrating a method of managing energy consumption by a wireless health monitoring device adapted for intermittent data transmission according to further aspects of the present disclosure.

FIG. 4 is a flowchart illustrating a method of transmitting data over a wireless network according to aspects of the present disclosure.

FIG. 5A is a diagram illustrating the use of a wireless health monitoring device adapted for intermittent data transmission according to aspects of the present disclosure.

FIG. 5B is a diagram illustrating the use of a wireless health monitoring device adapted for intermittent data transmission according to other aspects of the present disclosure.

FIG. 5C is a diagram illustrating the use of a wireless health monitoring device adapted for intermittent data transmission according to further aspects of the present disclosure.

### DETAILED DESCRIPTION OF EMBODIMENTS

As mentioned above, one solution for enabling mobile telemetry of health-related data involves a wearable health monitoring device that uses a low-power and/or short-range wireless technology to transmit health-related data to a separate local intermediating device that uses long-range wireless technology to relay the data to a remote destination, where diagnostics and/or clinical decision support algorithms are deployed, for example, to detect an exacerbation of a medical condition, such as an arrhythmia and/or suggest an intervention. Attempts at changing this configuration and developing solutions in the field of mobile telemetry of health-related data, however, has faced significant technical challenges, including limitations related to the constrained energy source of the proposed solutions.

Put another way, providing a wearable, mobile healthcare telemetry device that collects health-related data and reliably transmits that data directly to a remote destination (i.e., without requiring a local intermediating device as described herein) is a non-trivial task. For example, because the mobile telemetry device must be continuously worn by the patient for long periods of time, it may be inconvenient or impractical to frequently require removing the device for charging or replacing batteries. Further, because the device must overall be convenient and comfortable to wear and/or use, the on-board energy source(s) enclosed within such devices are constrained in both physical size and energy storage capacity. Accordingly, one of the technical challenges involved in this field is how to improve the lifetime of the device's constrained energy source such that it is feasible to transmit or upload health-related data from the monitoring device to support clinical decision-making without the use of a separate, local gateway device.

In accordance with the present disclosure, it is appreciated that for mobile health monitoring devices, such as the continuously operating telemetry devices described herein, energy consumption may be largely dominated by the communications technology (e.g., cellular communication, etc.) used to upload the data to the cloud. It is also appreciated that certain stages of a transmission/upload operation influence energy consumption more significantly than other stages (e.g., actually transmitting the data consumes more energy than establishing a wireless connection to a remote destination). Furthermore, it is appreciated that energy consumption of a data transmission via a wireless communications network strongly depends on the network coverage and condition of the network. Consequently, the energy source lifetime of the wearable device is heavily influenced by the coverage conditions it experiences, and therefore by the locations that its wearer visits while being monitored remotely. Specifically, when poor coverage is often experienced, it may drop below the minimally required lifetime.

As a result, when certain network conditions are observed (e.g., poor network conditions), the amount of energy consumed by the health monitoring device may exceed reserved amounts and cause the device to fall below its minimum lifetime-per-charge. In the conventional scenario, the burden of long-range data transmission was borne by the local gateway device, which could be conveniently charged without having to detach the monitoring device from the user. However, the health monitoring devices described herein perform the long-range data transmission and, in generally, cannot be charged without removing the device from the user and/or interrupting its monitoring functions. Thus, methods for improving energy consumption and health monitoring devices having improved energy consumption are desired.

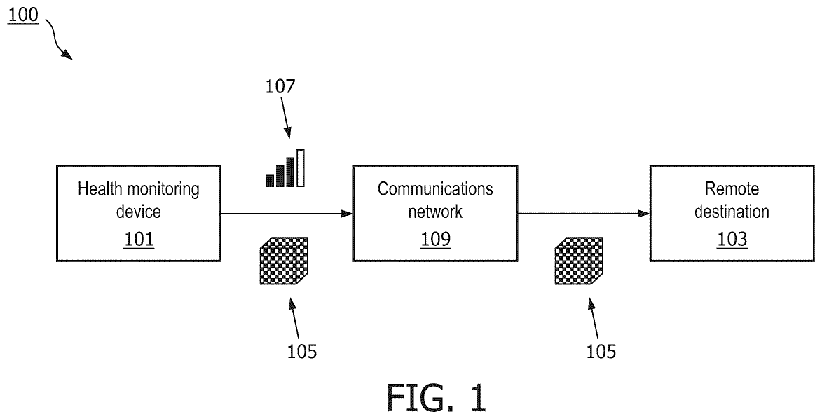

Accordingly, with reference to FIG. 1, a system 100 for improving the performance of a constrained energy source of a wireless health monitoring device 101 that is adapted for intermittent data transmission is illustrated according to aspects of the present disclosure. In embodiments, the system 100 includes the wireless health monitoring device 101 adapted for intermittent data transmission with controlled energy consumption. In further embodiments, the system 100 includes a remote destination 103, such as a remote server and/or cloud-based service. In embodiments, the system 100 operates by wirelessly transmitting health-related data 105 from the health monitoring device 101 using a long-range wireless signal to a communications network 109, which relays the health-related data 105 to the remote destination 103. The system 100 may not include and/or require a local intermediating device in order to complete the transmission of the health-related data 105 to the remote destination 103 and/or the communications network 109. As a result, in such embodiments, the health monitoring device 101 reduces costs by eliminating the additional intermediating device and improves usability of the monitoring solution by simplifying the solution without compromising on energy consumption. These and other technical improvements will be apparent to those of ordinary skill in the art based on the present disclosure.

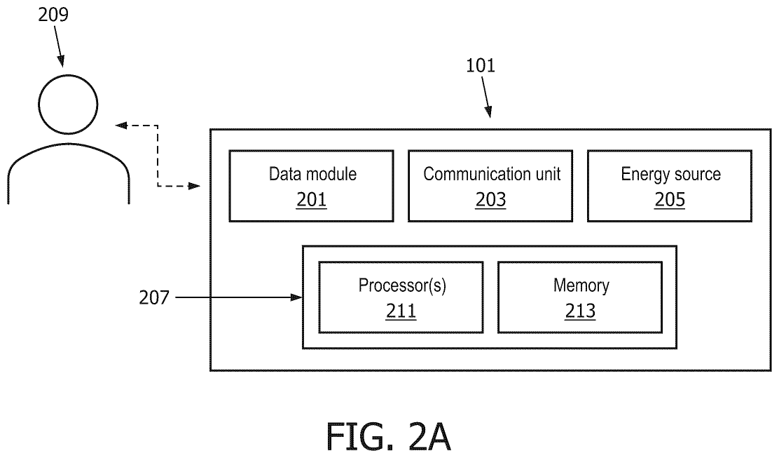

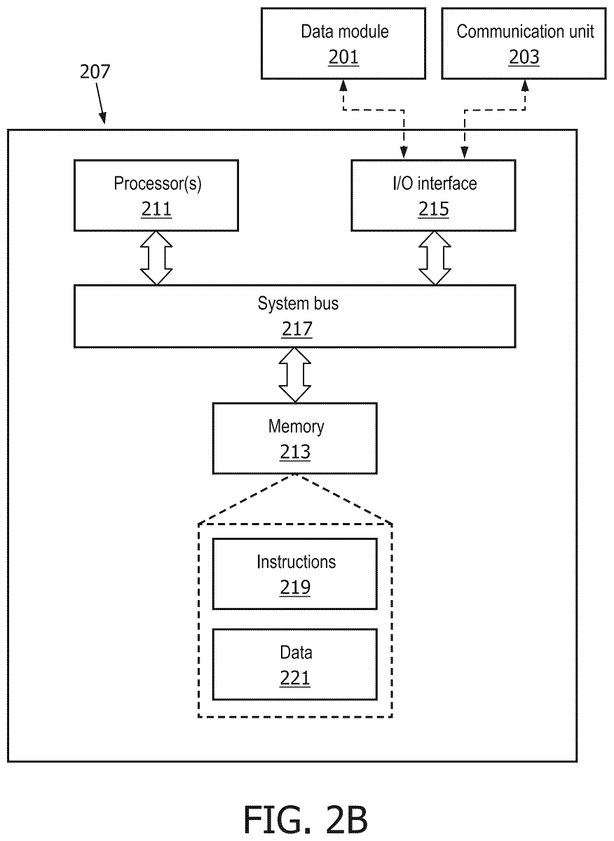

Turning to FIG. 2A, a wireless health monitoring device 101 adapted for intermittent data transmission with controlled energy consumption is illustrated according to aspects of the present disclosure. In embodiments, the wireless health monitoring device 101 includes a data module 201, a communication unit 203, a constrained energy source 205, and/or a controller 207. The wireless health monitoring device 101 may be configured to be worn by an individual 209. In some embodiments, for example, the wireless health monitoring device 101 may be attached to the skin of the individual 209, for example, configured as part of a patch having an adhesive backing. However, other means of wearing the device 101 are contemplated, including but not limited to, in the form of a ring, a wristband, a strap, a watch, and/or the like.

In embodiments, the data module 201 of the wireless health monitoring device 101 may be configured to generate and/or collect health-related data (e.g., health-related data 105) associated with at least one user 209. The data module 201 may be configured to generate and/or collect such health-related data over a period of time, such as several days to several months or more. Put another way, data module 201 may be configured to generate and/or collect health-related data (e.g., health-related data 105) over long periods of time. In embodiments, the period of time of monitoring the individual 209 is greater than the average charge cycle of the constrained energy source 205. In further embodiments, the period of time of monitoring the individual 209 is multiple times greater than the average charge cycle of the constrained energy source 205. In specific embodiments, the period of time of monitoring the individual 209 is at least 10 times the average charge cycle of the constrained energy source 205.

In embodiments, the data module 201 may include one or more physiological sensors associated with the user 209 such that the health-related data (e.g., health-related data 105) includes physiological sensor data collected by one or more of the physiological sensors. According to certain aspects of the present disclosure, a physiological sensor may employ an analog front end circuit that is configured to measure an analog physiological signal associated with the user 209, and an analog-to-digital converter that is operatively connected to the analog front end circuit and is configured to convert the analog physiological signal to digital sensor data. In specific embodiments, for example, the data module 201 may include a physiological sensor that is an electrocardiography (ECG) sensor configured to be worn by the user 209 and measure electrical signals on at least one area of the skin of the user 209. In such embodiments, the health-related data (e.g., health-related data 105) can include ECG sensor data.

In embodiments, the communication unit 203 of the wireless health monitoring device 101 may be operatively connected to the data module 201 and configured to wirelessly transmit health-related data (e.g., health-related data 105) over a network (e.g., communications network 109) to a remote destination (e.g., remote destination 103). As described herein, the communication unit 203 can be configured to wirelessly transmit health-related data directly to a long-range network that connects the wireless health monitoring device 101 directly to a remote destination 103, preferably without using an intermediating device local to the wireless health monitoring device 101 (such as a smartphone or mobile device within proximity to the health monitoring device 101) as a relay point for the health-related data.

In some embodiments, the communication unit 203 may include a modem configured to modulate one or more carrier signals in order to wirelessly transmit the health-related data over an appropriate long-range network. For example, in some embodiments, the communication unit 203 may be configured to modulate a carrier signal within one or more frequency bands, including one or more cellular frequencies, which allows the wireless health monitoring device 101 to connect to a wireless network 109, such as a cellular network. In particular embodiments, the communication unit 203 may utilize one or more of telecommunication standards, including but not limited to, LoRaWAN, NB-IoT, EC-GSM-IoT, LTE-M, LTE CAT1, SigFox, and/or the like. In some embodiments, the communication unit 203 may utilize one or more short and/or medium range wireless technologies in addition to a long-range wireless technology. For example, in some embodiments, the communication unit 203 may be further configured to utilize ZigBee, Z-Wave, Bluetooth, Bluetooth Low Energy, Wi-Fi HaLow, Thread, and/or the like.

In embodiments, the constrained energy source 205 of the wireless health monitoring device 101 may be operatively connected to one or more components of the health monitoring device 101 and configured to store and/or provide electrical energy thereto. For example, the constrained energy source 205 can be operatively connected to one or more of the data module 201, the communication unit 203, and/or the controller 207, and can be configured to store and/or provide electrical energy thereto.

In some embodiments, the constrained energy source 205 can include one or more battery cells, including one or more rechargeable battery cells. In specific embodiments, the constrained energy source 205 may include a lithium-ion battery cell. In other embodiments, the constrained energy source 205 can include one or more capacitors and/or ultracapacitors. In further embodiments, the constrained energy source 205 may include a combination of battery cells and capacitors or ultracapacitors.

In embodiments, the constrained energy source 205 has a complete discharge time of at least one day (i.e., 24 hours), including between two and ten days. In preferred embodiments, the constrained energy source 205 has a complete discharge time of about five days (i.e., 120 hours). As used herein, the term “complete discharge time” refers to the amount of time it takes to discharge the entire capacity of the constrained energy source 205 without having to recharge the energy source 205 and/or pause operation of the health monitoring device 101. Put another way, the term “complete discharge time” can refer to the amount of time the wireless health monitoring device 101 remains continuously operational before the constrained energy source 205 is depleted.

In embodiments, the wireless health monitoring device 101 also includes a controller 207 operatively connected to one or more components of the wireless health monitoring device 101, including one or more of the data module 201, the communication unit 203, and/or the energy source 205. In embodiments, the controller 207 includes one or more processors 211 and memory 213 configured to store machine-readable instructions for execution by the one or more processors 211. In particular embodiments, when the machine-readable instructions are executed by the one or more processors 211 of the controller 207, the health monitoring device 101 is caused to perform one or more steps of the methods/algorithms described herein.

With reference to FIG. 2B, a block diagram of the controller 207 is illustrated in accordance with further aspects of the present disclosure. As shown, the controller 207 can include one or more processors 211, machine-readable memory 213, an input/output (I/O) interface 215, all of which may be interconnected and/or communicate through a system bus 217 containing conductive circuit pathways through which instructions (e.g., machine-readable signals) and data may travel to effectuate communication, tasks, storage, and the like.

The one or more processors 211 may include one or more high-speed data processors adequate to execute the program components described herein and/or various specialized processing units as may be known in the art. In some examples, the one or more processors 211 may be a single processor, multiple processors, multiple processor cores on a single die, and/or the like, including combinations thereof.

The I/O interface 215 of the controller 207 may include one or more I/O ports that provide a physical connection to one or more devices outside of the controller 207. Put another way, the I/O interface 215 may be configured to connect, communicate, and/or control one or more associated devices. In some embodiments, the I/O interface 215 can include one or more serial ports. In embodiments, one or more components of the wireless health monitoring device 101 may be connected to the controller 207 via the I/O interface 215, including but not limited to the data module 201 and the communication unit 203. Put another way, the controller 207 may be connected to, communicate with, and control one or more components of the wireless health monitoring device 101 via the I/O interface 215.

The memory 213 of the controller 207 can be variously embodied in one or more forms of machine-accessible and machine-readable memory. In some examples, the memory 213 may include one or more types of memory, including one or more types of transitory and/or non-transitory memory. In particular embodiments, the memory 213 can include a magnetic disk storage device, an optical disk storage device, an array of storage devices, a solid-state memory device, and/or the like, including combinations thereof. For example, in specific embodiments, the memory 213 can comprise solid-state memory that includes volatile memory (RAM) and persistent memory (Flash memory).

As shown in FIG. 2B, the memory 213 can be configured to store machine-readable instructions 219 that, when executed by the one or more processors 211, cause the wireless health monitoring device 101 to perform one or more steps of the methods/algorithms described herein. In particular embodiments, the memory 213 may also be configured to store data 221 associated with performing the methods/algorithms described herein.

In embodiments, the controller 207 having been adapted to perform the processes described herein, may be provided in one or more software components, hardware components, and/or some combination of both software and hardware components. In embodiments, one or more of the software components may be incorporated into, loaded from, loaded onto, or otherwise operatively available to and from the controller 207. For example, in some embodiments, one or more software components of the controller 207 may be stored in the local memory 213. In further embodiments, one or more software components of the controller 207 may be loaded onto and/or updated from a remote server and/or cloud-based service (e.g., via the communication unit 203).

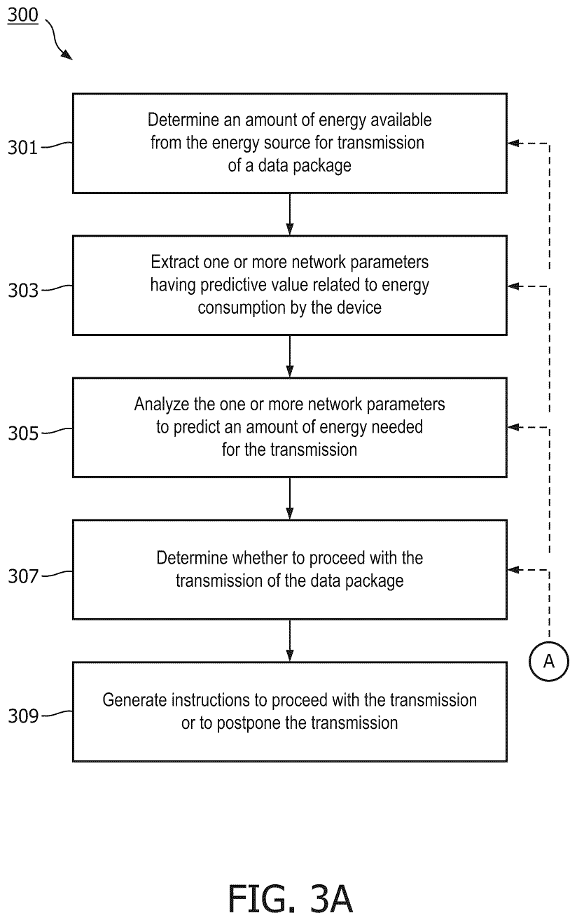

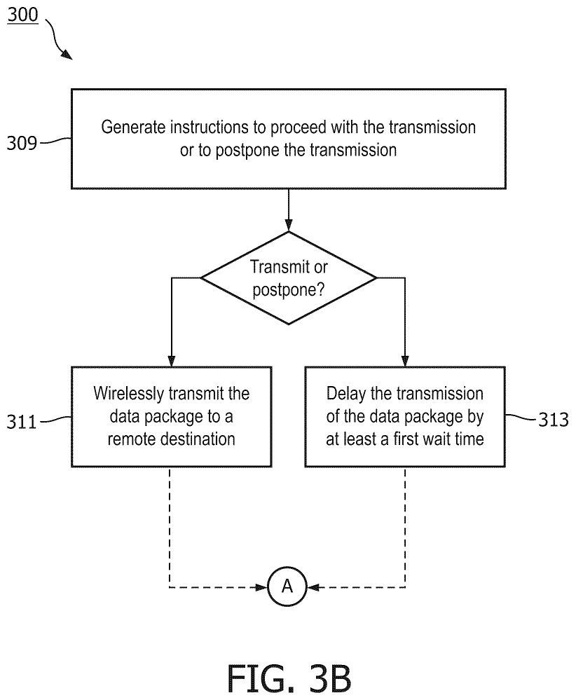

With reference to FIG. 3A, provided is a flowchart illustrating a method 300 of managing energy consumption by a wireless health monitoring device adapted for intermittent data transmission, including one or more of the processes to be performed and/or coordinated by the controller 207 based on the execution of machine-readable instructions 219 stored in the memory 213. In embodiments, the method 300 includes performing a pre-transmission assessment operation including one or more of the following steps: in a step 301, determining an amount of energy available from the constrained energy source 205 for transmission of a data package comprising health-related data 105; in a step 303, extracting one or more network parameters having predictive value related to energy consumption by the device 101 based on current network conditions; in steps 305 and 307, determine whether to proceed with the transmission of the data package based on the amount of energy available for the transmission and the one or more network parameters. In some embodiments, the method 300 further comprises: in a step 309, generating instructions to the one or more processors 211 to proceed with the transmission of the data package or to postpone the transmission of the data package. As shown in FIG. 3B, the method 300 can also include: in a step 311, wirelessly transmitting the data package from the device 101 via the communication unit 203 over the network 109 to a remote destination 103; and/or in a step 313, delaying the transmission of the data package from the device 101 by at least a first wait time.

As described in more detail later in this disclosure, in the method 300, the data transmission may be attempted periodically, i.e., where the first wait time involves a fixed amount of time elapsed relative to the currently attempted transmission. In other embodiments, the first wait time may be enlarged when the preceding data transmission was delayed, i.e., where the first wait time involves an increasing amount of time elapsed relative to the currently attempted transmission. The decision to enlarge the wait time depends on an estimation of the probability that network conditions have not improved since the preceding data transmission attempt, or vice versa where the time could be shortened when the probability that network conditions have been improved is estimated to be high. Such estimation of the probability that network conditions have or have not been improved may be based on vital sign, activity and posture measurements of the person wearing a device, for example, based on accelerometer and ECG data collected by the device 101.

More specifically, in the step 301, the method 300 can include determining an amount of energy available from an energy source (e.g., energy source 205) of a health monitoring device (e.g., device 101) for the transmission of a data package comprising health-related data (e.g., data 105). As described herein, the data package may include processed and/or un-processed health-related data that was collected via a data module (e.g., module 201) of the health monitoring device. That is, the health-related data collected by the data module of the health monitoring device may be processed, compressed, and/or otherwise formatted into a form suitable for transmission by a communication unit (e.g., unit 203). In embodiments, the health-related data may be processed, compressed, and/or otherwise formatted by one or more processors (e.g., processors 211) of the health monitoring device in accordance with instructions (e.g., instructions 219) stored in memory (e.g., memory 213).

In further embodiments, the data package may contain health-related data (e.g., data 105) that was generated and/or collected by the data module 201 over one or more periods of time. For example, in some embodiments, the health monitoring device 101 may be scheduled or programmed to attempt to upload health-related data 105 in five-minute increments; thus, each data package may comprise health-related data 105 collected over a previous five-minute period of time. In particular embodiments, the health-related data generated and/or collected by the data module 201 may be split into smaller data packages for uploading. The decision to divide an upload into multiple data packages as well as the desired size of each package may be determined based on the observed network conditions as determined by the one or more network parameters extracted in the step 305. For example, under progressively worse network conditions, progressively smaller data packages may be created for uploading. In further embodiments, one or more data packages may be combined into a larger data package that contains health-related data spanning multiple time intervals. For example, if transmission of one or more data packages is postponed (as described in more detail below), then those data packages may be combined into a larger data package for a single upload operation. The ultimate objective is to resolve the backlog in transmitting data packages as soon as the network conditions improve sufficiently relative to the available energy supply. However, until then, depending on the sensed network conditions, it may be optimal to divide the backlog into smaller data packages to maintain data transmission.

In embodiments, the amount of energy available from the energy source for transmission of each data package comprising health-related data (E_{upload}) may be fixed and/or predetermined based on the particular application. In other embodiments, E_{upload} may be calculated based on a total amount of energy available from the energy source (E_{batt}), which may be based on the current capacity of the energy source, i.e., the state of charge (SOC) of the energy source. That is, the amount of energy available from the energy source for an upload (E_{upload}) may be computed and recomputed each time an upload is performed based on the remaining charge (SOC) of the energy source and/or the remaining desired energy source lifetime.

In embodiments, the energy source may also be required to last for a minimum amount of time (T_{batt}) before it can be recharged or replaced, which may be set by commercial and/or clinical requirements, or may be adjusted based on user behavior (e.g., based on how frequently the user is in a position to charge the device or based on trends in the network conditions associated with the user's routine). More specifically, the minimum amount of time (T_{batt}) may be increased from a preset value, decreased from a preset value, and/or otherwise set based on a learned routine of the user's behavior.

In particular embodiments, the amount of power used by the health monitoring device for its functions other than transmission (Pₒₜₕₑᵣ) may be estimated or known, along with the total capacity (E_{batt}) of the energy source, the minimum usage period (T_{batt}), and the frequency of data uploads (f_{upload}), or its reciprocal the period of data uploads (T_{upload}). In embodiments, the amount of energy available for transmission of a data package (E_{upload}) may be a function of E_{batt}, Pₒₜₕₑᵣ, T_{batt}, and f_{upload}.

In specific embodiments, the amount of energy available for transmission of one or more data packages (E_{upload}) may be determined as follows:

$${E}_{upload} = \frac{{E}_{batt} - {P}_{other} * {T}_{batt}}{( {T}_{batt} / {T}_{upload} )} \qquad ( Eqn . 1 )$$

In some embodiments, the memory 213 of the controller 207 may include instructions 219 that, when executed by the one or more processors 211, determines the amount of energy available from the energy source 205 for transmission of a data package comprising health-related data (i.e., E_{upload}). For example, pseudo code for this operation using a fixed computation (i.e., once after full charge of the battery) and a dynamic computation (i.e., before each upload) is illustrated in Table 1 below:

```
TABLE 1
def get_max_battery_energy( ):
  # Function returns the maximum energy the battery can deliver [mWh]
  # ... calculation based on battery capacity and voltage.
  # ... example 500 mA * 3.7V = 1850 mWh
return max_battery_energy
def get_remaining_battery_energy( ):
  # Function returns the remaining energy the battery can deliver [mWh]
  # ... calculation based on for example discharge curve, coulomb meter
  # ... example 200 mAh 3.7V = 540 mWh
return remaining energy
def get_remaining_lifetime( desired_battery_lifetime ):
  # Function returns the remaining lifetime of the device
  # ... calculation based on desired_battery_lifetime and time the device has
been enabled
  # ... example 24h
  return remaining_lifetime
1. Fixed computation (once after full charge of the battery)
```

| | |
|---|---|
| power_consumption_other = 3 | # [mW] |

desired_battery_lifetime = 120 # [h]

| | |
|---|---|
| upload_freq = 12 | # number of uploads per hour |

```
batt_max_energy = get_max_battery_energy( ) # 1850
energy_consumption_other = power_consumption_other
desired_battery_lifetime # 3 * 120
available_energy = batt_max_energy − energy_consumption_other
    # 1850 − 360
upload_energy = available_energy / (desired_battery_lifetime * upload_freq)
  # 1490 / (120 * 12)
2. Dynamic computation (before each upload)
```

| | |
|---|---|
| power_consumption_other = 3 | # [mW] |
| desired_battery_lifetime = 120 | # [h] |
| upload_freq = 12 | # number of uploads per hour |

batt_current_energy = get_remaining_battery_energy( ) # 540
remaining_lifetime = get_remaining lifetime(desired_battery_lifetime) # 24
energy_consumption_other = power_consumption_other * remaining_lifetime
# 3 * 24
available_energy = batt_current_energy − energy_consumption_other
# 540 − 72
upload_energy = available_energy / (desired_battery_lifetime * upload_freq) #
468 / (24 * 12)

In particular embodiments, the amount of energy available from the energy source 205 for transmission of a data package including health-related data (i.e., E_{upload}) may be expressed per unit of data (e.g., per byte of data) to be transmitted. As described herein, this per unit of data expression of E_{upload} may be utilized in situations where, for example, the amount of data gathered over a period of time is variable or multiple periods of data gathering are bundled together in one data package.

In some embodiments, the total amount of energy available from the energy source (E_{batt}) may be the current total capacity of the energy source, which may be determined using a fuel gauge in one or more ways, including by measuring the voltage level of the energy source and comparing it to a discharge curve, by measuring the current using a Coulomb counter, and/or other similar methods.

In one or more embodiments, the maximum capacity of the energy source of the health monitoring device may range from about 500 mAh to about 2000 mAh at 3.7 volts, the power consumption of the non-transmission operations of the health monitoring device may be about 1 mA, the minimum battery time may range from about 1 day to about 5 days, and/or the upload frequency may range from about 6 to about 12 times per hour. However, it should be appreciated that the methods described herein may be implemented in other devices with other device specifications.

In the step 303, the method 300 can include extracting one or more network parameters having predictive value related to energy consumption by the health monitoring device (e.g., device 101) based on current network conditions. That is, the one or more processors 211 of the health monitoring device 101 may operate the communication unit 203 and extract one or more network parameters based on the current network conditions observed by the communication unit 203.

As described above, it is appreciated by the present disclosure that the energy consumed by the health monitoring device 101 during a data transmission via a long-range, wireless network (e.g., a cellular network) can be impacted by the coverage of the network. For example, it has been observed that the energy consumption of a data transmission via an LTE-M network can easily vary by more than an order of magnitude depending on the coverage situation encountered. In embodiments, network coverage can relate to a variety of parameters like signal strength (RSRP), signal quality (RSRQ, CINR), busyness of the network, and specific network settings imposed by the Mobile Network Operator (MNO). In specific embodiments, these network parameters can include, but are not limited to, a signal strength, a signal quality, a duration of a particular connection phase, and/or an energy consumption value for a particular connection phase.

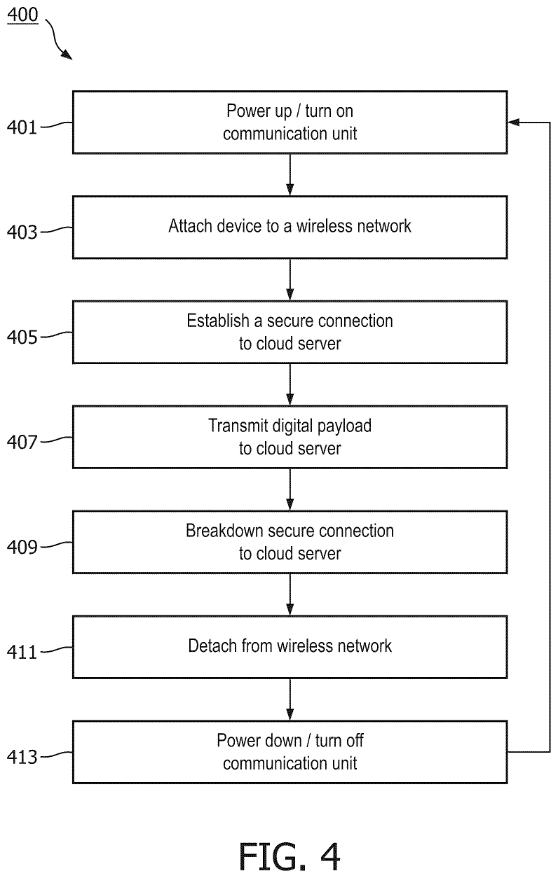

More specifically, with reference to FIG. 4, a method 400 of uploading data to an HTTP server (e.g., remote destination 103) over a cellular network (e.g. network 109) is illustrated according to aspects of the present disclosure. As shown, the method 400 includes: in a step 401, powering-up and/or turning on the communication unit (e.g., unit 203); in a step 403, attaching the device to a wireless network; in a step 405, establishing a secure IP (e.g., TLS or DTLS) connection to a cloud server; in a step 407, transmitting a digital payload to the cloud server and subsequently waiting for the corresponding response message (e.g., performing an HTTP post to transfer the payload and waiting for the corresponding HTTP response); in a step 409, breaking down the secure IP connection; in a step 411, detaching from the wireless network; and in a step 413, powering down and/or turning off the communication unit.

Although the process for uploading to an HTTP server over a cellular network is illustrated in FIG. 4 according to certain aspects of the present disclosure, it should be appreciated that similar processes can be performed for cloud servers using other IP protocols, such as CoAP over DTLS/UDP/IP, MQTT over TLS/TCP/IP, and QUIC/UDP/IP.

In embodiments, the step 303 can include extracting one or more network parameters having predictive value related to energy consumption by the health monitoring device during at least one of the attaching phase and the connecting phase of the method 400 (i.e., during step 403 and/or step 405). In some embodiments, the network parameters include at least one of a signal strength observed during the attachment phase, a signal quality observed during the attachment phase, the duration of the attachment phase, and/or an amount of energy from the energy source consumed during the attachment phase. In further embodiments, the network parameters includes at least one of a signal strength observed during the connection phase, a signal quality observed during the connection phase, the duration of the connection phase, and/or an amount of energy from the energy source consumed during the connection phase.

In some embodiments, the memory 213 of the controller 207 may include instructions 219 that, when executed by the one or more processors 211, extracts one or more network parameters having predictive value related to the energy consumption by a health monitoring device, which can include obtaining one or more parameters (e.g., signal strength, signal quality, and/or the like) from the communication unit 203. For example, pseudo code for this operation is illustrated in Table 2 below:

TABLE 2

tic = currenttime( )
AttachToNetwork( )
time_attach = currenttime( ) − tic
rsrp, rsrq = CollectMetrics( ) # network parameters
tic = currenttime( )
HttpConnect( )
time_connect = currenttime( ) − tic

Thus, in some embodiments, the one or more network parameters that are extracted in the step 303 are based on the observed network conditions prior to any transmission of health-related data. In additional and/or alternative embodiments, the step 303 may include uploading a small trial data package and extracting one or more network parameters in connection with the trial data package. For example, the step 303 may include the step 407 of the method 400 as shown in FIG. 4 in order to test the current network conditions, where the initial upload is a trial data package that is substantially smaller in size than the data package comprising the rest of the health-related data. As such, in certain embodiments, the network parameters extracted in the step 303 may also include at least one of a signal strength observed during a trial upload phase, a signal quality observed during the trial upload phase, the duration of the trial upload phase, and/or an amount of energy from the energy source consumed during the trial upload phase.

It should be appreciated that observing the network conditions during each of the phases as described herein are not mutually exclusive. That is, each of the network parameters at each of the phases described here may or may not have predictive value related to energy consumption by the health monitoring device depending on the overall circumstances. As such, for example, one or more network parameters observed from one phase may be included with one or more network parameters from one or more other phases and used in the next step 305. Additionally, in particular embodiments, one or more network parameters observed during a preceding upload (e.g., observed during a trial upload) may be extracted and used (alone or in combination with one or more subsequent network parameters) in the next step 305.

More particularly, in a step 305, the method 300 includes analyzing the one or more network parameters extracted in the step 303 in order to predict an amount of energy needed for the transmission of the data package (E_{predicted}). In some embodiments, a set of rules may be established to predict the amount of energy needed for the transmission based on the extracted network parameters. For example, the energy consumption of the health monitoring device may be correlated with signal strength and/or signal quality observed, as shown in Tables 3 and 4:

TABLE 3

| RSRP | Signal Strength | Energy Consumption |
|---|---|---|
| ≥−100 dBm | Good | Lowest |
| −100 dBm to −120 dBm | Fair | Middle |
| −120 dBm to −140 dBm | Poor | Highest |
| ≤−140 dBm | Very poor/No signal | N/A |

TABLE 4

| RSRQ | Signal Quality | Energy Consumption |
|---|---|---|
| ≥−10 dB | Excellent | Lowest |
| −10 dB to −15 dB | Good | Middle |
| −15 dB to −20 dB | Fair to poor | Highest |
| ≤−20 dB | No signal | N/A |

In further embodiments, the step 305 of the method 300 can include analyzing the extracted network parameters using a trained machine learning model. In embodiments, the machine learning model may be developed using a dataset comprising uploading statistics (e.g., network parameters) for a plurality of health monitoring devices, the associated energy consumption, the duration of the uploads (e.g., if energy consumption measurements are missing), and other performance-related data (e.g., number of charges, lifecycle of the energy sources, energy consumed per upload, etc.). The machine learning model may also be finetuned on the in-field collected data and/or finetuned to provide a reduced amount of false positives or false negatives, trading off sensitivity and specificity. Such finetuning of the predictive model can occur locally on the device and/or it can be performed by a cloud server and delivered to the device as a firmware update that can occur when appropriate. Updates may also involve federated learning techniques.

In embodiments, the trained machine learning model may comprise one or more of a support vector machine, a decision tree, a gradient boosted decision tree, a bagged tree, a logistic regression, a neural network, a k-nearest neighbors algorithm (KNN), and/or the like, including combinations thereof. In particular embodiments, the controller 207 may be configured to utilize one or more trained machine learning models (or multi-parameter statistical approaches) according to a hierarchy of different trained models. For example, depending on the amount of energy available from the constrained energy source, the controller 207 may analyze the one or more network parameters using a simpler, more energy efficient trained model first, and then escalating the use to more complex, less energy efficient, trained models. More specifically, if one or more network parameters are especially favorable (e.g., signal strength >−100 dBm), the data upload may be performed without needing to analyze the network parameters using a machine learning model. Similarly, if one or more network parameters are especially unfavorable (e.g., signal strength <−130 dBm), then the data upload may be delayed without needing to analyze the network parameters using a machine learning model.

In some embodiments, the trained machine learning model may be trained and fixed prior to deployment of the device 101. In other embodiments, the trained machine learning model may be updated and/or replaced during operation of a device 101. For example, in some embodiments, the controller 207 may receive, via the communication unit 203, one or more updated machine learning models from a remote server and/or cloud-based service. In further embodiments, the machine learning model may be adapted during operation of the device 101 to incorporate statistics related to current operational conditions (e.g., based on the current/real-time network parameters and actual battery consumption measurements).

In embodiments, the memory 213 of the controller 207 may include instructions 219 that, when executed by the one or more processors 211, analyze the one or more network parameters in order to predict an amount of energy needed for the transmission of a data package using a set of rules and/or a trained machine learning algorithm. In particular embodiments, for example, the set of rules and/or trained machine learning algorithm may receive at least one or more network parameters as inputs, and output an amount of energy likely needed for the transmission of the data package. In embodiments, the set of rules and/or trained machine learning algorithm may output a likelihood that transmission of the data package will consume a certain amount energy. For example, given a certain threshold (such as 1 mWh) where the method should transmit if the predicted energy is below and postpone if it above, the output will indicate a certain probability of it being below/above the threshold).

Next, in the step 307, the method 300 can include determining whether to proceed with the transmission of the data package. For example, in some embodiments, the step 307 may include determining whether the predicted amount of energy needed for transmission of the data package (E_{predicted}) is less than or equal to the amount of energy available for the upload (E_{upload}). In further embodiments, the step 307 may include determining whether the predicted amount of energy needed for transmission of the data package (E_{predicted}) is within a certain tolerance level set based on the amount of energy available for the upload (E_{upload}). That is, there may be an energy threshold that is set based on the amount of energy available for the upload (E_{upload}), which is used to compare with the predicted energy amount (E_{predicted}).

On the other hand, if the amount of energy predicted (E_{predicted}) is greater than the amount of energy available for the upload (E_{upload}) and/or the energy threshold set based on the upload energy (E_{upload}), then it may be determined that the transmission of the data package should be delayed (i.e., not transmitted until a later time when network conditions have changed).

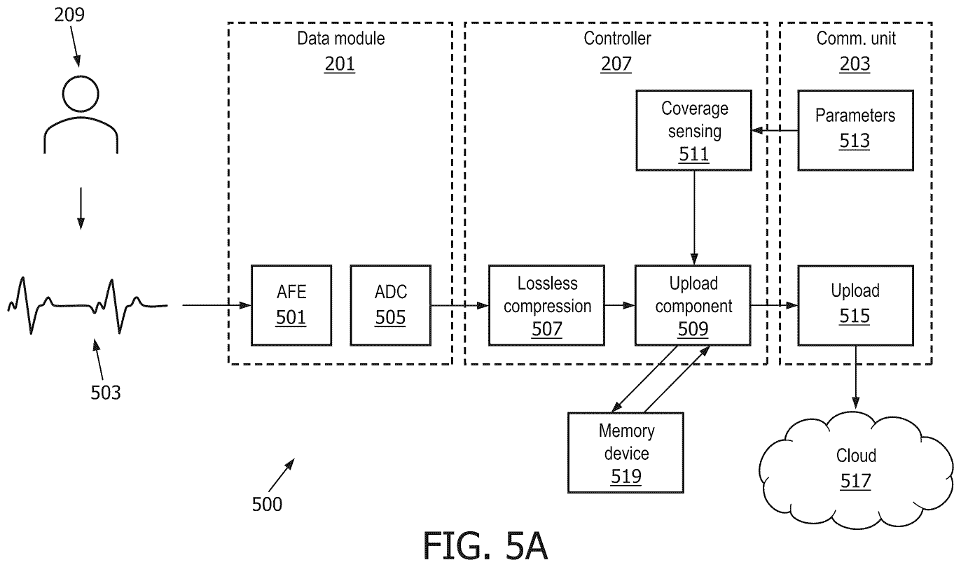

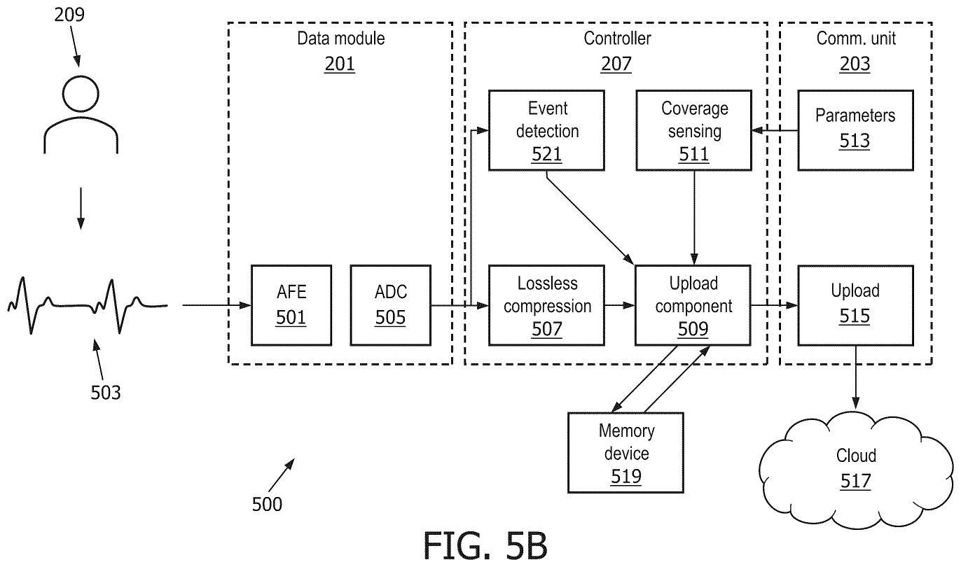

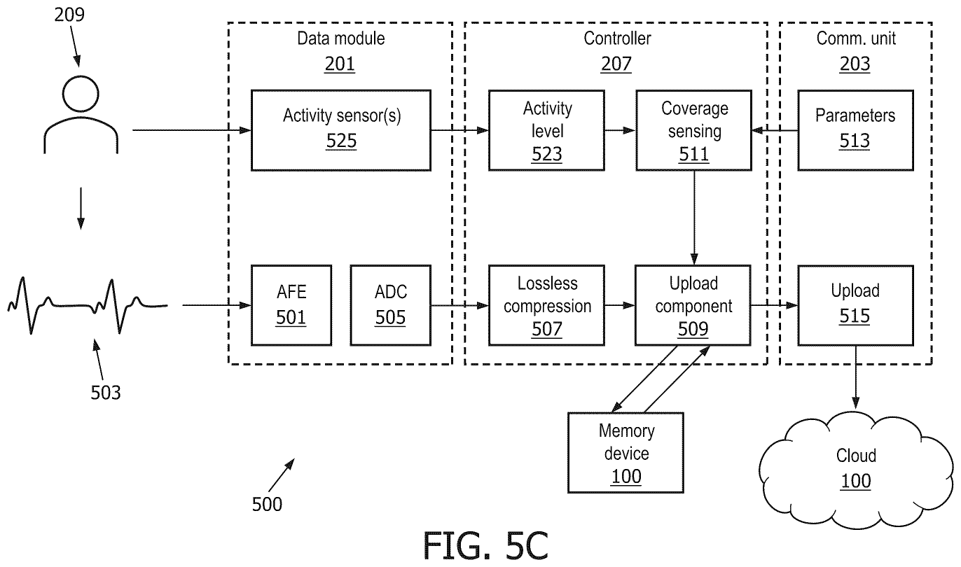

In some embodiments, the energy threshold for a particular data package may be based on the importance of the health-related data contained within the data package. For example, in some embodiments, the controller 207 may determine that the health-related data indicates the occurrence of an important or critical health-related event (discussed in more detail below with respect to FIG. 5B), and as a result, the energy threshold may be adjusted to enable uploading of the information regardless of the energy consumption. In other embodiments, the energy threshold may be adjusted such that a preset upload schedule is maintained. For example, the energy threshold may be adjusted to ensure that an upload or transmission occurs according to the preset schedule. In embodiments, this preset schedule may require that one or more uploads occur at least once per day, for example.

It should be appreciated that the step 307 can also include determining whether to proceed with the transmission of more than one data package. For example, as described herein, one or more data packages may have been delayed and/or postponed, and therefore there is a queue of two or more data packages waiting to be uploaded. In some embodiments, two or more data packages may be combined into a single data package for a single transmission operation. In such embodiments, the determination of whether to proceed with the upload may be based on whether the predicted amount of energy needed for the transmission of the combined data package (E_{predicted}) is less than or equal to an energy threshold that is set based on a corresponding amount of energy available for the upload (E_{upload}). In embodiments, the memory 213 of the controller 207 may include instructions 219 that, when executed by the one or more processors 211, determines whether perform an upload, such as according to the pseudo code pseudo code shown in Table 5:

```
TABLE 5
data_package_one = [....] # first health-related dataset
data_package_two = [....] # second health-related dataset
network_params = [....] # extracted network parameters
num_packages = 2 # variable based on queue of pending uploads
upload_energy = 1 mWh # amount of energy available for a single upload
combined_package = data_package_one.extend(data_package_two)
predicted_energy = predict_energy(num_packages, network_params)
if predicted_energy <= num_packages * upload_energy:
  upload(combined_package)
else:
  delay_upload( )
```

In other embodiments, instead of combining two or more individual data packages together, the step 307 can include determining whether to proceed with the transmission of at least one of the pending data packages. For example, if it is determined that the transmission should proceed, then the method 300 may continue to steps 309 and 311, at which point a first transmission operation may be performed for a first data package followed by a second transmission operation for another data package, and so on. These and other aspects are described in more detail below.

In embodiments, the determination of whether to transmit the data package may be adjusted or tailored to best suit the conditions of a particular device and/or its usage. For example, if a health monitoring device is being used by an individual in a community with overall poorer signal strength, that may be taken into account when determining whether to postpone a particular data package. On the other hand, if an individual's device has consistently good or excellent network conditions, that may also be taken into account.

In particular embodiments, step 305 and 307 may be combined in a single operation whereby the trained machine learning model predicts whether an upload should be performed or postponed without explicitly predicting an amount of energy required for the upload (E_{predicted}). That is, the trained machine learning model may combine the energy prediction and the threshold decision into a single step, which may be particularly advantageous if insufficient data is available to predict an exact amount of energy.

In the step 309, the method 300 then includes generating instructions to the one or more processors (e.g., processors 211) of the health monitoring device to either: (1) proceed with the transmission of the data package, or (2) postpone the transmission of the data package. In embodiments, if it is determined that the transmission should proceed, then the step 309 may include generating instructions to the one or more processors (e.g., processors 211) to proceed with the transmission of the data package. In other embodiments, if it is determined that the transmission should not proceed, then the step 309 may include generating instructions to the one or more processors (e.g., processors 211) to postpone the transmission of the data package.

In embodiments, the method 300 may further include transmitting and/or postponing the transmission of the data package. For example, as shown in FIG. 3B, the method 300 may include a step 311 comprising wirelessly transmitting the data package to a remote destination based on the instructions generated in step 309. In some embodiments, the duration of the upload operation and/or the amount of energy consumed during the upload operation may be monitored by the controller 207 via the communication unit 203. In such embodiments, the upload operation may be aborted if its energy consumption exceeds a predefined threshold and/or the duration exceeds a predefined threshold. This timeout is advantageous when the upload takes an unexpectedly long amount of time or an unexpectedly high amount of energy, for example, in case the prediction fails, because network conditions have deteriorated significantly during the data upload itself (e.g., when the patient wearing the device went into an area or a structure with poor cellular coverage).

As shown in FIG. 3B, the method 300 may also include a step 313 comprising delaying the transmission of the data package. In embodiments, the step 313 includes delaying or otherwise postponing the transmission of the data package by at least a first wait time. In some embodiments, the wait time can be a fixed amount of time that is predetermined for the device 101 based on the particular application. In further embodiments, the step 313 may include delaying or postponing the transmission of the data package according to a predetermined schedule. For example, as mentioned above, the health monitoring device 101 may be scheduled or programmed to attempt to upload health-related data 105 in five-minute increments; thus, the step 313 may postpone transmission of the data package until the next five-minute period arrives.

As mentioned above, if there are multiple pending data packages prepared for individual transmission, then one or more steps of the method 300 may be repeated. For example, in some embodiments, if it is unlikely that network conditions have changed (e.g., little time has passed since a prior upload, etc.), then the method 300 may return to step 307 and determine whether to upload another data package. In other embodiments, the method 300 may return to step 301, 303, and/or 305 as well, depending on the changes in network conditions, timing of the uploads, activity of the user, changes in the energy source, and/or the like. In further embodiments, the network parameters observed during a preceding upload may be utilized to determine whether a subsequent upload should be performed.

Turning now to FIG. 5A, a systematic diagram illustrating the use of a wireless health monitoring device 500 is shown in accordance with certain implementations and embodiments of the present disclosure. In the example of FIG. 5A, the health monitoring device 500 comprises a data module 201 operatively connected to a controller 207, and a communication unit 203. The data module 201 comprises an analog front-end 501 configured to measure an analog physiological signal 503 associated with an individual 209, and an analog-to-digital converter 505 configured to convert the analog physiological signal into a digital signal (i.e., health-related data). The controller 207 may include a compression 507 component configured to compress the digital signal in a lossless compression operation. The controller 207 may include an upload component 509 configured to determine whether to transmit one or more data packages containing health-related data (e.g., the compressed data output by the compression component 507). The upload component 509 may receive one or more network parameters extracted by a coverage sensing component 511 of the controller 507. Put another way, the coverage sensing unit 511 and the upload component 509 may be configured to perform one or more steps of the methods 300, 400 described herein. In particular embodiments, the coverage sensing component 511 may use the communication unit 203 to determine one or more network parameters 513, which are provided to the upload component 509 as described above.

If the upload component 509 determines that a data package comprising health-related data should be transmitted to the cloud (i.e., a remote server, etc.), the communication unit 203 may perform an upload operation 515, which wirelessly transmits the data package over a communications network to a remote location 517, such as a remote server and/or cloud-based service.

In embodiments, the health monitoring device 500 also includes a memory storage device 519, which may be configured to store the health-related data until it is transmitted off of the health monitoring device 500. In particular embodiments, the memory storage 519 may be a component of the controller 207 (e.g., as a part of memory 213), or may be a dedicated storage device.

With reference to FIG. 5B, a systematic diagram illustrating the use of a wireless health monitoring device 500 is shown in accordance with additional implementations and embodiments of the present disclosure. In the example of FIG. 5B, the health monitoring device 500 comprises a data module 201 operatively connected to a controller 207 and a communication unit 203, as described above. However, the controller 207 shown in FIG. 5B further comprises an event detection component 521 configured to analyze the health-related data received from the data module 201. For example, in certain embodiments, the data module 201 may be configured to detect an ECG signal 503 from the user 209, which the event detection component 521 of the controller 207 may analyze to determine whether any important cardiovascular events (arrhythmias, etc.) are indicated.

In embodiments, the event detection component 521 may provide instructions to the upload component 509 indicating that a significant physiological event has taken place. Based on those instructions from the event detection component 521, the upload component 509 may determine that transmission of the health-related data should proceed regardless of the pre-transmission energy assessment. That is, for example, the step 309 of the method 300 may include generating instructions to the one or more processors to proceed with the transmission of the data package even if the predicted amount of energy is greater than an energy threshold. Put another way, the energy threshold may be eliminated based on the instructions received from the event detection component 521 that a significant and/or potentially significant physiological event is or has taken place.

With reference to FIG. 5C, a systematic diagram illustrating the use of a wireless health monitoring device 500 is shown in accordance with still additional implementations and embodiments of the present disclosure. In the example of FIG. 5C, the health monitoring device 500 comprises a data module 201 operatively connected to a controller 207 and a communication unit 203, as described above. However, the controller 207 further comprises an activity level component 523 and the data module 201 further comprises one or more activity sensors 525 that the activity level component 523 may use to determine whether to re-check the current network conditions and/or attempt another data transmission operation. In particular, it is appreciated that checking network conditions and predicting energy usage according to the present disclosure are steps which also consume energy. Accordingly, in particular embodiments, the decision to perform such steps may be based on a determined likelihood that network conditions have changed.

In embodiments, the one or more activity sensors 525 may be configured to measure one or more activity indicators, including but not limited to, additional vital signs of the user 209, the activities of the user 209, the posture of the user 209, the position/location of the user 209, and/or the like, in order to estimate the probability that the current network conditions have changed. That is, the activity level component 523 of the controller 207 may receive these one or more activity indicators from the activity sensors 525 and estimate the probability that the current network conditions (e.g., signal strength, signal quality, etc.) have changed.

For example, in some embodiments, if the activity indicators from the activity sensors 525 indicate that the user 209 has not moved locations or changed positions since the controller 207 last evaluated the current network conditions, the activity level component 523 may determine that it is unlikely that the current network conditions have changed. Alternatively, if the activity indicators from the activity sensors 525 indicate that the user 209 has moved locations or changed positions since the controller 207 last evaluated the current network conditions, the activity level component 523 may determine that it is likely that the current network conditions have changed.

In embodiments, the activity indicators may also include data collected from the data module 201, such as ECG information. In specific embodiments, the activity sensors 525 may include compasses, pressure sensors, GPS sensors, ECG sensors, an accelerometer, and/or the like.

In embodiments, one or more steps of the methods described herein (e.g., method 300) may be omitted based on the estimated likelihood that the current network conditions have changed. For example, where one or more network parameters were analyzed (e.g., in a step 305) and a transmission operation was performed (e.g., in a step 311) because a positive determination was made (i.e., network conditions were good), if the activity level component 525 indicates that it is unlikely that network conditions have changed since that evaluation, then another transmission operation (e.g., in another step 311) may be performed for another data package containing health-related data without re-evaluating the current network conditions. In other embodiments, the controller 207 may re-evaluate network conditions and/or optionally re-evaluate network conditions before performing another transmission operation. For example, certain determinations may be made according to the decision chart illustrated in Table 6 below:

TABLE 6

| | | Likelihood of Change in Network Conditions | | |
|---|---|---|---|---|
| | | Likely | Neutral | Unlikely |
| Preceding | Good | Re-evaluate | Optionally re- | Skip re- |
| Network | | | evaluate | evaluation |
| Evaluation | Neutral | Re-evaluate | Optionally re- | Optionally re- |
| | | | evaluate | evaluate |
| | Bad | Re-evaluate | Optionally re- | Skip re- |
| | | | evaluate | evaluation |

In particular embodiments, if the current network conditions are evaluated as ‘poor’ and the controller 207 finds that it is unlikely that network conditions have changed, then the controller 207 may progressively increase the duration between when the network conditions are next evaluated. For example, in some embodiments, if a first upload is delayed (e.g., at a step 313 of the method 300) due to poor network conditions, the controller 207 may wait until the next scheduled upload time to re-evaluate the network conditions and make another determination on whether to proceed with the upload. However, in further embodiments, if a first upload is delayed due to poor network conditions and it is unlikely that network conditions have changed (i.e., based on the determination of the activity level component 525), then the controller 207 may delay one or more future uploads until it is more likely that network conditions have changed (i.e., based on the determination of the activity level component 525).

Thus, for example, if a health monitoring device (e.g., device 101) is normally configured to attempt an upload operation every five minutes, the time between each attempt may be extended to 10 minutes, 20 minutes, 30 minutes, and so on when poor network conditions are detected and conditions have likely not changed. Similarly, in other embodiments, if a health monitoring device is normally configured to attempt an upload operation every five minutes, multiple pending uploads may be performed on a shortened time frame when good network conditions are detected and conditions have likely not changed.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A wireless health monitoring device adapted for intermittent data transmission with controlled energy consumption, the device comprising:
a data module configured to generate and/or collect health-related data associated with at least one user over a period of time;
a communication unit operatively connected to the data module, the communication unit being configured to wirelessly transmit health-related data over a network to a remote destination;
a constrained energy source operatively connected to the data module and the communication unit for storing and providing electrical energy thereto;
a controller operatively connected to the data module, the constrained energy source, and the communication unit, wherein the controller comprises a memory storing machine readable instructions and one or more processors that, when executing the machine-readable instructions, are configured to perform a pre-transmission assessment operation comprising one or more of the following:
i. determine whether a data package comprising the health-related data is ready to be transmitted from the device;
ii. determine an amount of energy available for the transmission of the data package within the constrained energy source;
iii. extract one or more network parameters having predictive value related to energy consumption by the device based on current network conditions; and
iv. determine whether to proceed with the transmission of the data package based on the amount of energy available for the transmission and the one or more network parameters.

2. The wireless health monitoring device of claim 1, wherein the data module comprises a physiological sensor associated with the user and wherein the health-related data comprises physiological sensor data collected by the physiological sensor.

3. The wireless health monitoring device of claim 2, wherein the physiological sensor comprises an electrocardiography sensor configured to be worn by the user.

4. The health monitoring device of claim 1, wherein the constrained energy source comprises a battery.

5. The health monitoring device of claim 1, wherein the one or more processors are further configured to wirelessly transmit the data package from the device via the communication unit over the network or to delay the transmission based on an outcome of the pre-transmission assessment operation.

6. The health monitoring device of claim 5, wherein the pre-transmission assessment operation further comprises predicting an amount of energy necessary for the transmission of the data package based on the one or more network parameters; and wherein an outcome of the pre-transmission assessment operation comprises generating instructions to the one of more processors to proceed with the transmission of the data package when the predicted amount of energy is less than or equal to an energy threshold, or to delay the transmission of the data package when the predicted amount of energy exceeds the energy threshold,
wherein the energy threshold is based at least in part on the amount of energy available for the transmission of the data package within the constrained energy source.

7. The health monitoring device of claim 6, wherein the energy threshold is further based at least in part on an acuity level of the health-related data in the data package.

8. The health monitoring device of claim 6, wherein the amount of energy necessary for the transmission of the data package is predicted based on the one or more network parameters using a trained machine learning model, the trained machine learning model comprising one or more of a support vector machine, a decision tree, a gradient boosted decision tree, a bagged tree, a logistic regression, and a neural network.

9. The health monitoring device of claim 1, wherein the one or more processors are configured to extract the one or more network parameters having predictive value related to energy consumption by the device based on current network conditions by:
connecting the health monitoring device to the wireless network via the communication unit; and
determining the one or more parameters based on the network conditions detected after connecting the health monitoring device to the wireless network;
wherein the one or more parameters comprise one or more of a signal strength, a signal quality, a duration of the connecting step, and an amount of energy consumed by the device during the connecting step.

10. The health monitoring device of claim 1, wherein the transmission readiness determination for the data package comprising the health-related data is based on one or more predetermined conditions, and the one or more processors are further configured to activate the communication unit and initiate the pre-transmission assessment operation when the one or more predetermined conditions are met.

11. A method of managing energy consumption by a wireless health monitoring device adapted for intermittent data transmission and comprising a data module, a controller having a memory and one or more processors, a communication unit, and a constrained energy source, the method comprising:
determining, via the one or more processors of the controller, an amount of energy available from the energy source for transmission of a data package comprising health-related data;
extracting, via the communication unit and the one or more processors of the controller, one or more network parameters having predictive value related to energy consumption by the device based on current network conditions;
analyzing, via the one or more processors of the controller, the one or more network parameters to predict an amount of energy needed for the transmission of the data package using a trained machine learning model;
determine whether to proceed with the transmission of the data package; and
generating instructions to the one or more processors to proceed with the transmission of the data package when the predicted amount of energy is less than or equal to an energy threshold, or to postpone the transmission of the data package when the predicted amount of energy exceeds the energy threshold,
wherein the energy threshold is based at least in part on the amount of energy available for the transmission of the data package within the constrained energy source.

12. The method of claim 11, further comprising wirelessly transmitting the data package to a remote destination, or delaying the transmission of the data package by at least a first wait time.

13. The method of claim 11, the trained machine learning model comprises one or more of a support vector machine, a decision tree, a gradient boosted decision tree, a bagged tree, a logistic regression, and a neural network.

14. The method of claim 11, wherein the step of extracting the one or more network parameters having predictive value related to energy consumption by the device based on current network conditions comprises:
connecting the health monitoring device to a wireless network via the communication unit; and
determining the one or more parameters based on the network conditions detected after connecting the health monitoring device to the wireless network;
wherein the one or more parameters comprise one or more of a signal strength, a signal quality, a duration of the connecting step, and an amount of energy consumed by the device during the connecting step.

15. The method of claim 14, wherein the step of determining the one or more parameters comprises transmitting a portion of the data package via the wireless network and detecting the network conditions during said transmission.
